(19) United States
(12) Patent Application Publication
(10) Pub. No.: US 2025/0381102 A1
(43) Pub. Date: Dec. 18, 2025

(54) COOLING APPLICATORS FOR COSMETIC PRODUCTS

(71) Applicant: HCT GROUP HOLDINGS LIMITED, Sai Wan (CN)

(72) Inventor: Denis Maurin, Los Angeles, CA (US)

(73) Assignee: HCT GROUP HOLDINGS LIMITED, Sai Wan (CN)

(21) Appl. No.: 19/232,194

(22) Filed: Jun. 9, 2025

Related U.S. Application Data
(60) Provisional application No. 63/659,044, filed on Jun. 12, 2024.

(51) Int. Cl. A61K 8/02

(52) U.S. Cl. CPC A61K 8/0229; A61K 2800/87

(57) ABSTRACT

An applicator device includes a housing with a reservoir and an applicator tip assembly. The applicator tip assembly includes a main body portion and a metal applicator. The main body has an upper face with an annular groove extending inward and radially between an outer wall of the main body portion and an inner wall. The inner wall defines a product delivery passageway fluidly coupled to an aperture in the metal applicator. The metal applicator is coupled to the main body portion and has an application face and an annular protrusion extending from a face opposite the application face, where the annular protrusion is configured to engage the annular groove of the main body portion with a friction fit. The metal applicator has a first outer diameter at the application face and a second, smaller outer diameter at an end opposite the application face.

### CROSS REFERENCE TO RELATED APPLICATIONS

The present application claims the benefit of U.S. Provisional Patent Application Ser. No. 63/659,044, filed Jun. 12, 2024, the disclosure of which is incorporated herein by reference.

### BACKGROUND

Devices exist for dispensing cosmetic or medicinal products. Such devices usually consist of an outer tubular shell or housing, a delivery mechanism for displacement of the cosmetic or medicinal products, and an applicator tip. For example, in the medical industry, applicators are employed for applying medicinal products, such as ointments, to portions of the body. In the cosmetics and personal care industries, applicators are used to apply lipstick, lip balm, skin creams, lotions, serums, and other cosmetic products to portions of the body.

In many cases, these medicinal and cosmetic products may include skin care substances, such as aloe or lanolin, that provide a healing or therapeutic effect to heal damaged skin or maintain healthy skin. In addition, these products may include therapeutic substances, such as topical anesthetics, analgesics, fragrances, menthol, or other substances that provide a soothing or stimulating sensation when applied to skin of a user of the product. In addition to skin care substances, thermal treatments (e.g., application of heat and/or cold) are known to relieve pain, provide a therapeutic sensation, and to slow the body's natural response to injury so that a slower and more controlled healing process may ensue. There is an ongoing need to provide alternative application devices and improved applicator devices.

### SUMMARY

This summary is provided to introduce concepts of applicators and applicator devices with applicator tip assemblies, which are further described below in the Detailed Description. This summary is not intended to identify essential features of the claimed subject matter, nor is it intended for use in determining the scope of the claimed subject matter. An example applicator device includes a housing having a reservoir for containing a cosmetic or medicinal product, and an applicator tip assembly including a main body portion having a first end coupled to the housing and a second end including an upper face having an annular groove extending inward toward the second end, the annular groove extending radially between an outer wall of the main body portion and an inner wall, the inner wall defining a product delivery passageway configured to convey the cosmetic or medicinal product, and a metal applicator coupled to the main body portion and having an aperture defined therein, the metal applicator having an application face for applying the cosmetic or medicinal product to a surface, and an annular protrusion extending from a face opposite the application face, the annular protrusion configured to engage the annular groove of the main body portion with a friction fit, the metal applicator having a first outer diameter at the application face and a second, smaller outer diameter at an end opposite the application face, wherein the product delivery passageway of the main body portion is fluidly coupled to the aperture in the metal applicator.

Alternatively or additionally to the embodiment above, the metal applicator defines a radially outwardly extending flange configured to extend over and cover an upwardly facing surface of the outer wall of the second end of the main body portion.

Alternatively or additionally to any of the embodiments above, the upper face of the main body portion is angled relative to a longitudinal axis of the housing.

Alternatively or additionally to any of the embodiments above, the annular protrusion of the metal applicator includes a radially outward extending ridge configured to engage a recess in an inner surface of the outer wall.

Alternatively or additionally to any of the embodiments above, the annular protrusion has a friction fit with at least one of the outer walls and the inner wall.

Alternatively or additionally to any of the embodiments above, the application face has a teardrop shape with the aperture positioned off-center.

Alternatively or additionally to any of the embodiments above, the main body portion comprises a thermoplastic polymer.

Alternatively or additionally to any of the embodiments above, the thermoplastic polymer comprises polypropylene.

Alternatively or additionally to any of the embodiments above, the main body portion and the housing are formed as a single piece.

Alternatively or additionally to any of the embodiments above, the application face is generally convex.

Alternatively or additionally to any of the embodiments above, the applicator tip assembly is fixed to the housing against movement relative to the housing.

Alternatively or additionally to any of the embodiments above, the product delivery passageway of the main body portion ends at a lower surface of the aperture through the metal applicator.

Alternatively or additionally to any of the embodiments above, the applicator device further comprises a cap configured to removably engage the applicator tip assembly, the cap including a plug that extends into and seals the product delivery passageway when the cap is disposed on the applicator tip assembly.

Another example applicator device comprises a housing having a reservoir for containing a cosmetic or medicinal product, and an applicator tip assembly comprising a main body portion having a first end coupled to the housing and a second end opposite the first end, the second end including an outer circumferential wall and an inner post spaced apart from the outer circumferential wall by an annular groove, the inner post defining a product delivery passageway to convey the cosmetic or medicinal product from the reservoir through the main body portion, and a metal applicator coupled to the main body portion, the metal applicator having an application face for applying the cosmetic or medicinal product to a surface, the metal applicator having an aperture extending therethrough, wherein the aperture is positioned over the inner post and forms a secondary product delivery passageway aligned with the product delivery passageway in the inner post, the metal applicator having a radially extending flange extending over and completely covering the outer circumferential wall of the main body portion.

Alternatively or additionally to the embodiment above, the metal applicator includes an annular extension extending away from the application face and configured to be received within the annular groove of the main body portion.

Alternatively or additionally to any of the embodiments above, an outer wall of the annular extension of the metal applicator includes a radially outward extending ridge configured to engage a recess in an inner surface of the outer circumferential wall.

Alternatively or additionally to any of the embodiments above, an outer surface of the inner post includes a radially outward extending ridge configured to engage an inner wall of the annular extension.

Alternatively or additionally to any of the embodiments above, the annular extension has a friction fit with both the outer circumferential wall and an outer surface of the inner post.

Alternatively or additionally to any of the embodiments above, the second end of the main body portion is angled relative to a longitudinal axis of the housing.

A further example applicator device comprises a housing having a reservoir for containing a cosmetic or medicinal product, and an applicator tip assembly comprising a main body portion coupled to the housing, the main body portion having a first end coupled to the housing and a second end opposite the first end, the second end defining a tubular product delivery passageway surrounded by an annular channel and an outer wall spaced apart from the tubular product delivery passageway by the annular channel, and a metal applicator including an application face and an opposing annular wall, the annular wall configured to surround and engage the tubular product delivery passageway in a friction fit, the annular wall configured to be disposed within the annular channel of the main body portion, the metal applicator including a radially outwardly extending flange configured to cover an end face of the outer wall of the main body portion, the application face having an aperture extending therethrough, wherein an upper end of the tubular product delivery passageway fluidly engages a lower end of the aperture.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items.

FIG. 1 is a perspective exploded view of an example applicator device with applicator tip assembly according to one embodiment;

FIG. 2 is a front cross-sectional view taken along line A-A of FIG. 1;

FIG. 3 is the view of FIG. 2 with the cap removed;

FIG. 4 is an enlarged exploded partial view of FIG. 3;

FIG. 5 is a side view of the applicator device of FIG. 1 with the cap removed;

FIG. 6 is a perspective exploded view of another example applicator device with applicator tip assembly according to another embodiment;

FIG. 7 is a front cross-sectional view taken along line a-a of FIG. 6;

FIG. 8 is the view of FIG. 7 with the cap removed;

FIG. 9 is an enlarged exploded partial view of FIG. 8;

FIG. 10 is a side view of the applicator device of FIG. 6 with the cap removed;

FIG. 11 is a perspective exploded view of a further example applicator device with applicator tip assembly according to a further embodiment;

FIG. 12 is a front cross-sectional view taken along line b-b of FIG. 11;

FIG. 13 is the view of FIG. 12 with the cap removed; and

FIG. 14 is an exploded front cross-sectional view of the applicator device of FIG. 11.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

The terms “monolithic” and “unitary” shall generally refer to an element or elements made from or consisting of a single one-piece structure. A monolithic and/or unitary element shall exclude structure and/or features made by assembling or otherwise joining multiple discrete elements together.

It is noted that references in the specification to “an embodiment”, “an example”, “some embodiments”, “some examples”, etc., indicate that the embodiment(s) or example(s) described may include a particular feature, structure, or characteristic, but every embodiment or example may not necessarily include the particular feature, structure, or characteristic. Moreover, such phrases are not necessarily referring to the same embodiment or example. Further, when a particular feature, structure, or characteristic is described in connection with a particular embodiment or example, it would be within the knowledge of one skilled in the art to affect the particular feature, structure, or characteristic in connection with other embodiments or examples, whether or not explicitly described, unless clearly stated to the contrary. That is, the various individual elements described below, even if not explicitly shown in a particular combination, are nevertheless contemplated as being combinable or arrangeable with each other to form other additional embodiments or examples, or to complement and/or enrich the described embodiment(s) or example(s), as would be understood by one of ordinary skill in the art.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

This disclosure is directed to applicator devices with applicator tip assemblies with a metal applicator configured to transfer and/or store and maintain a level of thermal energy. In some embodiments, due to the choice of materials and the relative size of the metal applicator, the metal applicator has the ability to convey a sensation of warming or cooling, and can regenerate that ability without external heating or cooling, other than exposure to ambient conditions. The metal applicator defines an application surface and an aperture, through which product may be dispensed. A product may be dispensed from the applicator device through the applicator tip assembly for application to a surface such as, for example, a user's skin. By virtue of the thermal retention or transfer of the metal applicator, thermal energy may be applied to the dispensed product so that it may be heated or cooled during application. Moreover, the application face of the metal applicator may transfer heat to or from the user's skin, thereby causing the user to feel a thermal sensation (warm or cool depending on the thermal energy in or transferred via the thermal storage applicator portion). In some cases, the heat or cold transfer may also minimize or alleviate pain or discomfort caused by damage to the skin or other surface.

Suitable metals may include, but are not limited to aluminum, titanium, steel, nickel, tin, copper, brass, platinum, alloys thereof. In some embodiments, the alloy ZAMAK (or ZAMAC) may be used. In others, stainless steel may be used.

The applicator tip assembly may be coupled to a housing such as a tube, bottle, tottle or other container for storing product. The main body of the applicator tip assembly may be monolithically formed with the housing, or made separately and coupled to the housing such as by injection molding. In some embodiments, the applicator tip assembly may be made separate of the housing and coupled thereto in a later operation.

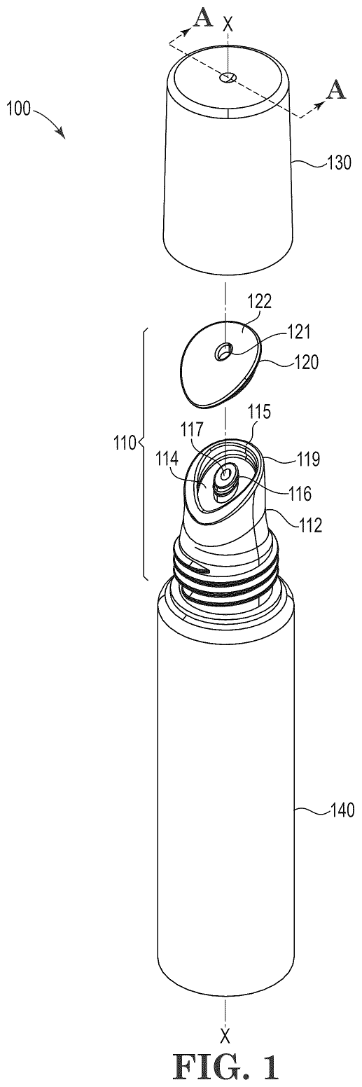

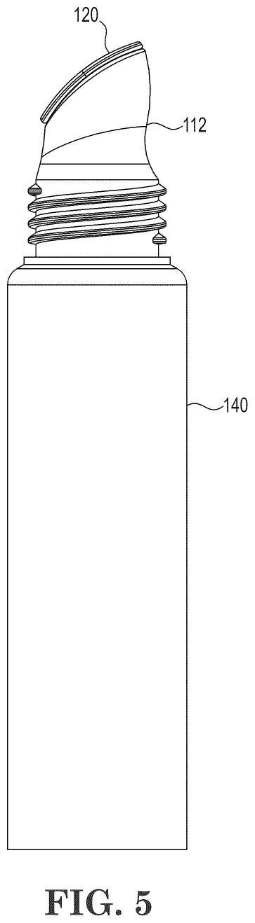

FIG. 1 represents an illustrative applicator device 100 with an applicator tip assembly 110, a cap 130, and a housing 140. In this embodiment, the applicator tip assembly 110 includes a main body portion 112 coupled to the housing 140 and a metal applicator 120 coupled to the main body portion. The metal applicator 120 defines an application face 122 surrounding an aperture 121. When product is dispensed through the aperture 121 from within the housing 140, the application face 122 may be used to apply or spread the product on the user's skin. In some embodiments, the metal applicator 120 and the application face 122 may have a teardrop shape and the aperture 121 may be off-center, as shown in FIG. 1. In other embodiments, the metal applicator 120 and the application face 122 may be circular, oblong, oval, or any other shape with a curved outer perimeter. The application face 122 may be generally convex, as shown in FIGS. 1 and 5. In other embodiments, the application face may be substantially flat. In the embodiments shown in the figures, the overall shape of the metal applicator 120 and the application face 122 have similar shapes. Alternatively, the application face 122 may have a different shape than the remainder of the metal applicator 120. For example, the application face 122 may be a convex teardrop shape as shown in FIG. 1, while the remainder of the metal applicator 120 may be circular, oval, square, or any other shape.

The metal applicator 120 may be made of a metal such as, for example, stainless steel or ZAMAC alloy. In other embodiments, the metal applicator 120 may comprise zinc, aluminum, magnesium, titanium, steel, nickel, tin, copper, brass, platinum, or alloys thereof, including ZAMAK. When the metal applicator 120 is made of stainless steel, for instance, it may have a mass from about 5 to about 15 grams. According to certain other embodiments, metal applicators 120 may have a mass of at most about 20 grams regardless of the metal. In some embodiments, metal applicators 120 may have a mass of about 1 gram to about 50 grams, from about 2 grams to about 40 grams; and/or about 5 grams to about 25 grams. The metal applicator 120 may have a volume from about 0.1 centimeters³ to about 0.5 centimeters³. However, according to still other embodiments, metal applicators 120 according to this disclosure may have masses and/or volumes smaller or larger than those listed above.

The main body portion 112 may have a first end coupled to the housing 140 and a second end including an upper face 119 having an annular groove 114 extending inward toward the second end, the annular groove 114 extending radially between an outer wall 115 of the main body portion and an inner wall 116. The outer wall 115 may be a circumferential wall defining a continuous outer perimeter at the second end of the main body portion 112. The upper face 119 may have the same shape as the application face 122 of the metal applicator 120. The inner wall 116 may define a product delivery passageway 117 configured to convey the cosmetic or medicinal product from the housing 140 through the aperture 121 in the metal applicator 120. The inner wall 116 may be in the form of a tubular post with a lumen defining the tubular product delivery passageway 117. The metal applicator 120 is coupled to the main body portion 112 with the aperture 121 extending completely through the metal applicator 120. In some embodiments, the upper face 119 of the main body portion 112 may be angled relative to a longitudinal axis x-x of the housing 140, as shown in FIG. 1.

The main body portion 112 may be made of a thermoplastic polymer, such as, for example, polypropylene, which is non-reactive with the product stored in the reservoir 142. In other implementations, the main body portion 112 may be made of plastic, metal, glass, or any other suitable material.

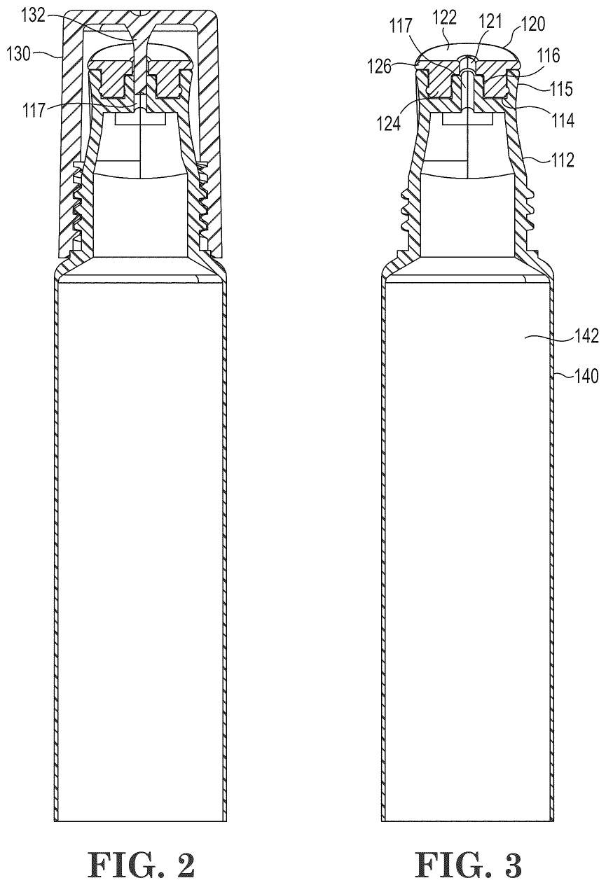

FIGS. 2-4 illustrate elements of the example applicator device 100 from a cross-sectional view along section line A-A of FIG. 1. The section line A-A extends through the widest part of the metal applicator 120 as seen from the front. The applicator device 100 may also include a cap 130 configured to removably engage and cover the applicator tip assembly 110 when the applicator device 100 is not in use and includes a plug 132 that extends into and seals the product delivery passageway 117 when the cap is disposed on the applicator tip assembly. See FIG. 2. The plug 132 may be made of a thermoplastic polymer similar to the main body portion 112 of the applicator tip assembly 110 or any other material which is non-reactive or resistant to the product being dispensed, such as various metals, plastics, ceramics, composites, or the like. Additionally, or alternatively, either the plug 132, the main body portion 112, or both may be elastomeric, such that when the cap is in place either the plug 132, the main body portion 112, or both, may expand and deform somewhat to seal the product delivery passageway 117. The cap 130 is configured to couple to the housing 140, such as by use of a snap-fit attachment, a threaded attachment as shown in FIG. 2, a press or friction fit, magnet(s), one or more hinges, or any other suitable means of attachment. As shown in FIG. 2, the cap 130 may have an inner thread that threadingly mates with an outer thread on the first end of the main body portion 112.

The housing 140 and main body portion 112 can be fabricated by molding as a single piece, or by the use of an insert molding process to both create and attach the main body portion to the housing 140. In some examples the housing 140 is a single layer material, while in others the housing 140 may be a laminated or other multi-layer structure. The housing 140 may be an extruded piece having one or more layers, such as using a two-layer design with an inner layer selected for compatibility to product that will be contained and dispensed by the device, and an outer layer selected for ease of decoration and/or visual appearance, or to facilitate attachment to the main body portion 112. If needed or desired, additional layers may be included to, for example, aid in securing different materials of inner and outer layers to one another (such as having a tie layer).

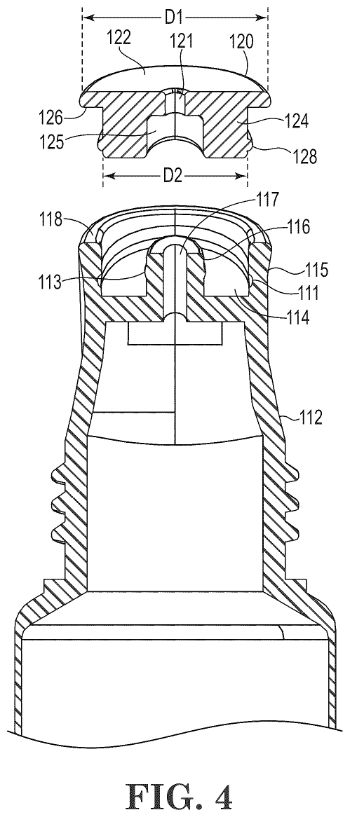

As shown in FIGS. 3 and 4, the metal applicator 120 may have an application face 122 for applying the cosmetic or medicinal product to a surface, and an annular extension, wall, or protrusion 124 extending from a face opposite the application face. The annular protrusion 124 may be configured to be received in and engage the annular groove 114 of the main body portion 112 with a friction fit. In other words, the annular wall or protrusion 124 surrounds and engages the post or inner wall 116 defining the product delivery passageway in a friction fit. When the annular protrusion 124 is fully seated within the annular groove 114, the product delivery passageway 117 of the main body portion 112 is fluidly coupled to the aperture 121 in the metal applicator 120 to convey the cosmetic or medicinal product from the reservoir 142 defined by the housing 140 to exit the application face 122 of the metal applicator 120. In the embodiment shown in FIGS. 2-3, the aperture 121 through the application face 122 is positioned over the inner wall 116 or post to form a secondary product delivery passageway aligned with the product delivery passageway 117 such that the upper end of the product delivery passageway 117 ends at and abuts the lower surface of the aperture 121 through the metal applicator 120.

The front cross-sectional view of the metal applicator 120 and upper part of the main body portion 112 in FIG. 4 illustrates details of the structures connecting the metal applicator 120 to the main body portion 112. As shown in FIG. 1, the metal applicator 120 has a teardrop shape with the narrowest portion being the lower edge as seen from the front. The cross-sectional line extends through the widest part of the metal applicator and the main body portion 112. The metal applicator 120 may have a first outer dimension D1 at the widest point (width) adjacent the application face 122, and a second, smaller outer dimension D2 at the widest point (width) at an end opposite the application face. D1 may be in a range of 9 mm to 15 mm and D2 may be in a range of 6 mm to 10 mm. In some examples, D1 may be 9 mm, 10 mm, 11 mm, 11.5 mm, 12 mm, or 13 mm with D2 being 7 mm, 7.5 mm, 8 mm, 8.5 mm, 9 mm or 9.5 mm respectively. In some embodiments, the difference between D1 and D2 may define a radially outwardly extending flange 126 configured to extend over and completely cover an upwardly facing surface 118 of the outer wall 115 of the second end of the main body portion. The lower side of the flange 126 that engages the upwardly facing surface 118 of the outer wall 115 may have a radially extending length (D1-D2) of 2 mm to 5 mm, with some embodiments being 3 mm. The flange 126 of the metal applicator 120 covering the entire upwardly facing surface 118 of the main body portion may provide a metal surface around the entirety of the application end of the applicator device 100.

The annular protrusion 124 may be configured to have a friction fit with both the outer wall 115 and the inner wall 116. In some embodiments, the annular protrusion 124 of the metal applicator 120 may include a radially outward extending ridge 128 configured to engage a recess 111 in an inner surface of the outer wall 115 of the main body portion 112. See FIG. 4. The engagement of the ridge 128 within the recess 111 may provide a snap-fit of the metal applicator 120 with the main body portion 112. The ridge 128 may extend circumferentially around the annular protrusion 124. Alternatively, the ridge 128 may be discontinuous and include two or more sections of radially outward extending ridges 128 spaced apart around the annular protrusion 124. In one embodiment, two ridges 128 may be disposed on opposing sides of the annular protrusion. The recess 111 may extend continuously and circumferentially around an inner surface of the outer wall 115. In other embodiments, the recess 111 may include separate, discrete sections corresponding to the positions of the sections of radially outward extending ridges 128. In some embodiments, the outer surface of the inner post or inner wall 116 may have a radially outward extending ridge 113 configured to engage the inner wall 125 of the annular extension. The ridge 113 may be a continuous circumferential ridge 113 and the inner wall 125 of the annular extension may be substantially flat such that the ridge 113 engages the inner wall 125 in a friction fit. In other embodiments, the inner wall 125 may include a recess (not shown) sized and positioned to receive the ridge 113.

While engagement of the metal applicator 120 to each of the outer wall 115 and inner wall 116 is illustrated in FIGS. 2-3, other designs can be used. In some examples, the metal applicator may have a friction fit only relative to the outer wall 115, and the inner wall 116 may have a smooth outer surface sized for easy passage into the metal applicator 120 without friction fit. In some other examples, the metal applicator may have a friction fit only relative to the inner wall 116, and the outer wall 115 may have a smooth inner surface sized for easy passage around the metal applicator 120 without friction fit. It may be noted that the seam between the metal applicator 120 and outer wall 115 is positioned along the side of the main body/device, and is recessed relative to the actual application surface. This means that a user will not feel the transition between the metal applicator 120 and outer wall 115 when applying a product using the application face 122. This enhances comfort during use, and makes the fit-up between parts at that seam less sensitive to variation.

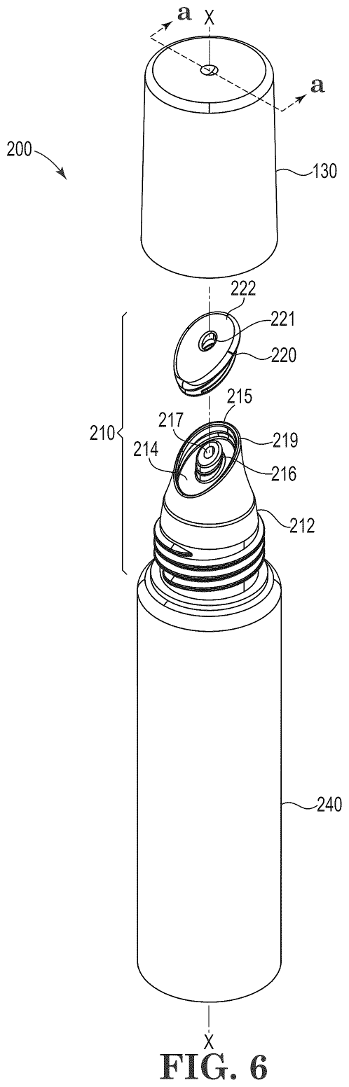

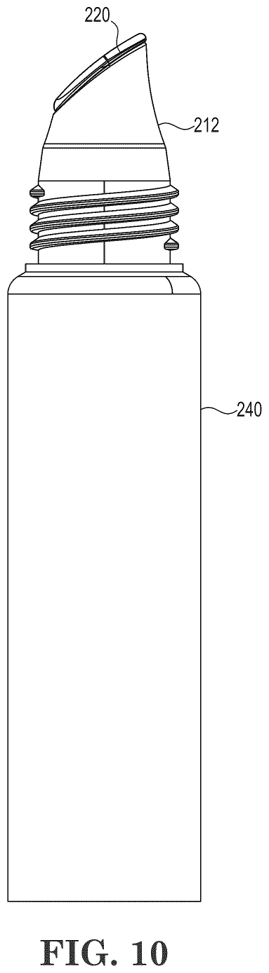

FIGS. 6-10 illustrate a variation of the embodiment shown in FIGS. 1-5, with differences in the main body portion 212 and metal applicator 220. FIG. 6 represents another applicator device 200 with an applicator tip assembly 210, housing 240, and cap 130. The cap 130 may be the same as that used with the main body portion 112 described above. In this embodiment, the applicator tip assembly 210 includes a main body portion 212 coupled to the housing 240 and a metal applicator 220 coupled to the main body portion. The metal applicator 220 may have an application face 222 with an ovoid shape and an off-center aperture 221. The application face 222 may be generally convex, as shown in FIGS. 6 and 10. The metal applicator 220 may be made of a metal including all of the metals and alloys described above for metal applicator 120. The mass and/or volume of the metal applicator 220 may be the same as that of the metal applicator 120 described above, or they may be smaller.

The main body portion 212 may have a first end coupled to the housing 240 and a second end including an upper face 219 having an annular recess or groove 214 extending inward toward the second end, the annular groove 214 extending radially between an outer wall 215 of the main body portion and an inner post or wall 216. The outer wall 215 may be a circumferential wall defining a continuous outer perimeter at the second end of the main body portion 212. The inner wall 216 may define a product delivery passageway 217 configured to convey the cosmetic or medicinal product from the housing 240 to the metal applicator 220. The metal applicator 220 is coupled to the main body portion 212 and has an aperture 221 extending completely through the metal applicator 220. In some embodiments, the upper face 219 of the main body portion 212 may be angled relative to a longitudinal axis x-x of the housing 240, as shown in FIG. 6.

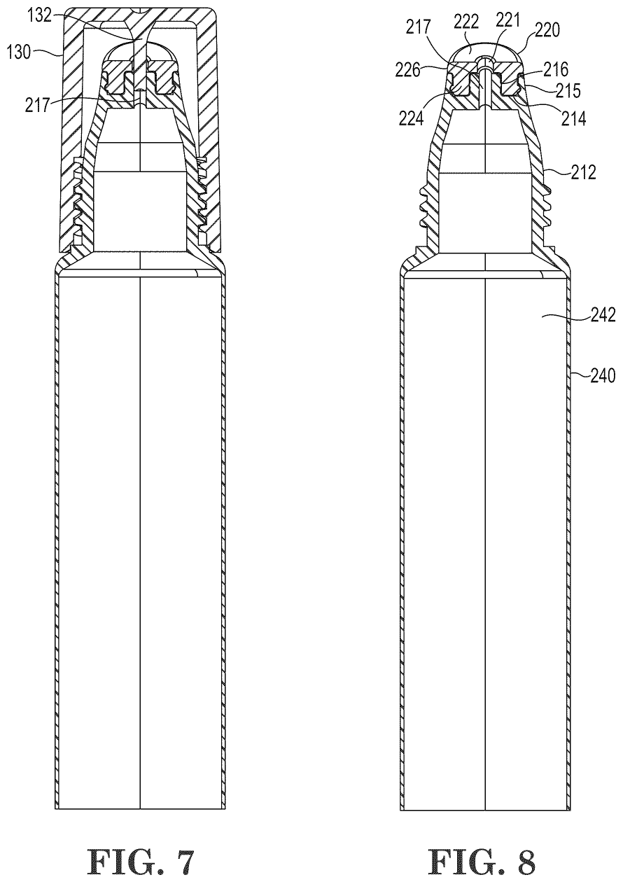

FIGS. 7-9 illustrate elements of the example applicator device 200 from a cross-sectional view along section line a-a of FIG. 6 which extends through the widest part of the metal applicator 220 and main body portion 212 as seen from the front. The applicator device 200 may also include a cap 130 configured to removably engage and cover the applicator tip assembly 210 when the applicator device 200 is not in use and includes a plug 132 that extends into and seals the product delivery passageway 217 when the cap is disposed on the applicator tip assembly. See FIG. 7. The plug 132 may be made of a thermoplastic polymer similar to the main body portion 212 of the applicator tip assembly or any other material which is non-reactive or resistant to the product being dispensed, such as various metals, plastics, ceramics, composites, or the like. Additionally, or alternatively, either the plug 132, the main body portion 212, or both may be elastomeric, such that when the cap is in place either the plug 132, the main body portion 212, or both, may expand and deform somewhat to seal the product delivery passageway 217. The cap 130 is configured to couple to the housing 240, such as by use of a snap-fit attachment, a threaded attachment as shown in FIG. 7, a press or friction fit, one or more hinges, or any other suitable means of attachment. As shown in FIG. 7, the cap 130 may have an inner thread that threadingly mates with an outer thread on the first end of the main body portion 212.

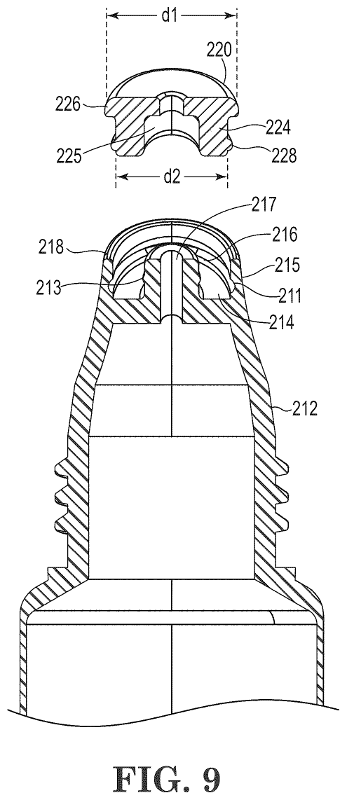

As shown in FIGS. 8 and 9, the metal applicator 220 may have an application face 222 for applying the cosmetic or medicinal product to a surface, and an annular extension, wall, or protrusion 224 extending from a face opposite the application face. The annular protrusion 224 may be configured to be received in and engage the annular channel or groove 214 of the main body portion 212 with a friction fit. In other words, the annular wall or protrusion 224 surrounds and engages the post or inner wall 216 defining the product delivery passageway in a friction fit. When the annular protrusion 224 is fully seated within the annular groove 214, the product delivery passageway 217 of the main body portion 212 is fluidly coupled to the aperture 221 in the metal applicator 220 to convey the cosmetic or medicinal product from the reservoir 242 defined by the housing 240 to exit the application face 222 of the metal applicator 220. In the embodiment shown in FIGS. 8 and 9, the aperture 221 through the application face 222 is positioned over the inner wall 216 or post to form a secondary product delivery passageway aligned with the product delivery passageway 217 such that the upper end of the product delivery passageway 217 ends at and abuts the lower surface of the aperture 221 through the metal applicator 220. The main body portion 212 may be made of a thermoplastic polymer, such as, for example, polypropylene, which is non-reactive with the product stored in the reservoir 242. In other implementations, the main body portion 212 may be made of plastic, metal, glass, or any other suitable material. As shown in FIG. 9, the cross-sectional line extends through the widest part of the metal applicator 220 and the main body portion 212. The metal applicator 220 may have a first outer dimension d1 at the widest point (width) adjacent the application face 222, and a second, smaller outer dimension d2 at the widest point (width) at an end opposite the application face. In some embodiments, the metal applicator 220 may define a radially outwardly extending flange 226 configured to extend over and completely cover an upwardly facing surface 218 of the outer wall 215 of the second end of the main body portion.

In some embodiments, d1 may be in a range of 6 mm to 15 mm and D2 may be in a range of 5 mm to 9 mm. In some examples, d1 may be 7 mm, 8 mm, 9 mm, 10 mm, or 12 mm with d2 being 6 mm, 7 mm, 7.5 mm, 8 mm, or 8.5 mm, respectively. In some embodiments, the difference between d1 and d2 may define a radially outwardly extending flange 226 configured to extend over and completely cover an upwardly facing surface 218 of the outer wall 215 of the second end of the main body portion. The lower side of the flange 226 that engages the upwardly facing surface 218 of the outer wall 215 may have a radially extending length (d1-d2) of 1 mm to 3 mm, with some embodiments being 1.5 mm.

The annular protrusion 224 is configured to have a friction fit with both the outer wall 215 and the inner wall 216. In some embodiments, the annular protrusion 24 of the metal applicator 120 may include a radially outward extending ridge 228 configured to engage a recess 211 in an inner surface of the outer wall 215 of the main body portion 212. See FIG. 9. Then engagement of the ridge 228 with the recess 211 may provide a snap-fit of the metal applicator 220 with the main body portion 212. The ridge 228 may extend circumferentially around the annular protrusion 224. Alternatively, the ridge 228 may be discontinuous and include two or more sections of radially outward extending ridges 228 spaced apart around the annular protrusion 224. In one embodiment, two ridges 228 may be disposed on opposing sides of the annular protrusion. The recess 211 may extend continuously and circumferentially around the inner surface of the outer wall 215. In other embodiments, the recess 211 may include separate, discrete sections corresponding to the positions of the sections of radially outward extending ridges 228. In some embodiments, the outer surface of the inner post or wall 216 may have a radially outward extending ridge 213 configured to engage the inner wall 225 of the annular extension. The ridge 213 may be a continuous circumferential ridge 213 and the inner wall 225 of the annular extension may be substantially flat such that the ridge 213 engages the inner wall 225 in a friction fit. In other embodiments, the inner wall 225 may include a recess (not shown) sized and positioned to receive the ridge 213.

While engagement of the metal applicator 220 to each of the outer wall 215 and inner wall 216 is illustrated in FIGS. 7-8, other designs can be used. In some examples, the metal applicator 220 may have a friction fit only relative to the outer wall 215, and the inner wall 216 may have a smooth outer surface sized for easy passage into the metal applicator 220 without friction fit. In some other examples, the metal applicator may have a friction fit only relative to the inner wall 216, and the outer wall 215 may have a smooth inner surface sized for easy passage around the metal applicator 220 without friction fit. It may be noted that the seam between the metal applicator 220 and outer wall 215 is positioned along the side of the main body/device, and is recessed relative to the actual application surface 222. This means that a user will not feel the transition between the metal applicator 220 and outer wall 215 when applying a product using the application face 222. This enhances comfort during use, and makes the fit-up between parts at that seam less sensitive to variation.

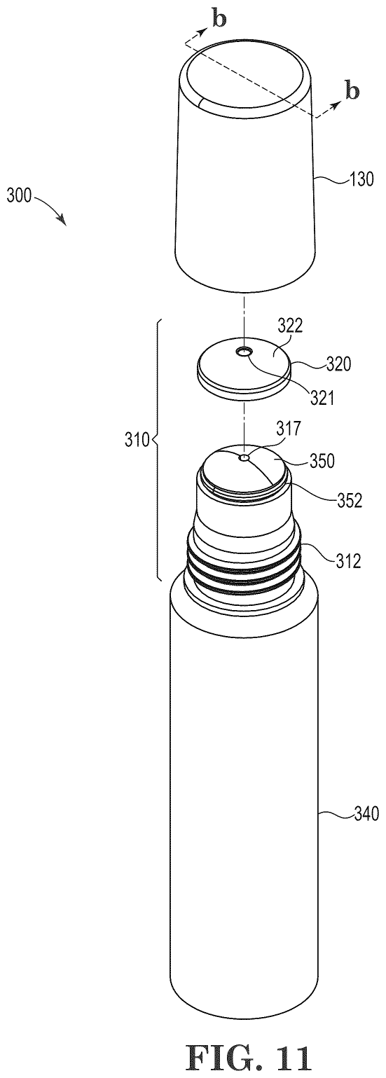

FIGS. 11-14 illustrate another embodiment of applicator device 300 with a different applicator structure. FIG. 11 represents an illustrative applicator device 300 with an applicator tip assembly 310, a cap 130, and a housing 340. The cap 130 may be the same as that used with the main body portion 112 described above. In this embodiment, the applicator tip assembly 310 includes a main body portion 312 coupled to the housing 340 and a metal applicator 320 coupled to the main body portion. The metal applicator 320 may have an application face 322 with a substantially circular shape and a centered aperture 321. In other embodiments, the application face 322 may be teardrop, oblong, oval, or any other shape with a curved outer perimeter. The application face 322 may be generally convex. In other embodiments, the application face may be substantially flat. The metal applicator 320 may be made of a metal such as, for example, stainless steel or ZAMAC alloy as described above for the metal applicator 120. The metal applicators 320 according to this disclosure may have masses and/or volumes smaller or larger than those listed above.

The main body portion 312 may have a first end coupled to the housing 340 and a second end including an upper face 350 having an aperture 317 extending into an interior of the main body portion 312 and an annular groove 352 extending radially inward around a perimeter of the upper face 350. The annular groove 352 may be a continuous, circumferential groove, as shown in FIG. 11. In other embodiments, the annular groove 352 may include a plurality of discontinuous grooves distributed around the perimeter of the upper face 350. The aperture 317 may define a product delivery passageway 317 configured to convey the cosmetic or medicinal product from the housing 340 to the metal applicator 320. The metal applicator 320 is coupled to the main body portion 312 and has an aperture 321 defined therein that extends completely through the metal applicator 320.

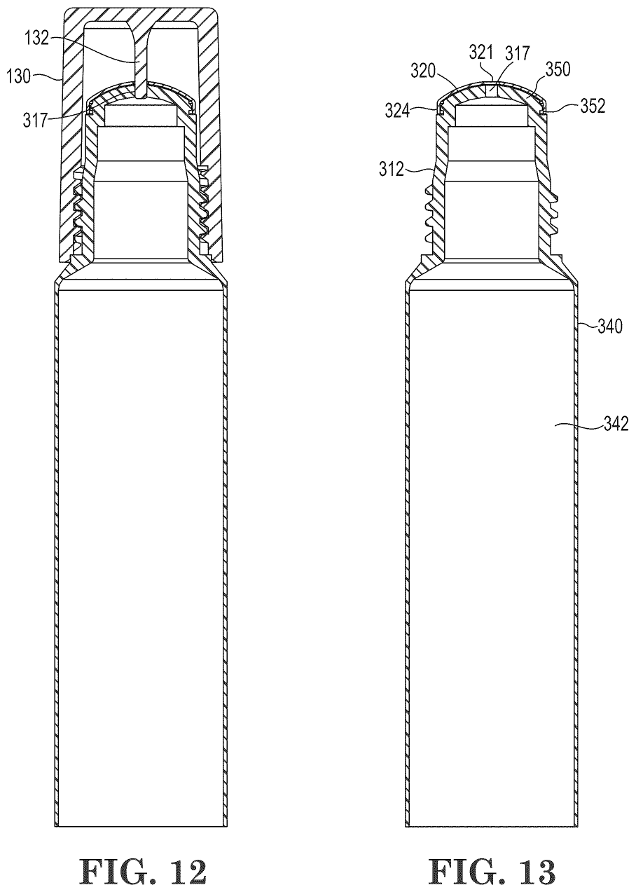

FIGS. 12-14 illustrate elements of the example applicator device 300 from a cross-sectional view along section line b-b of FIG. 11. The applicator device 300 may also include a cap 130 configured to removably engage and cover the applicator tip assembly 310 when the applicator device 300 is not in use and includes a plug 132 that extends into and seals the product delivery passageway 317 when the cap is disposed on the applicator tip assembly. See FIG. 132. The plug 132 may be made of a thermoplastic polymer similar to the main body portion 312 of the applicator tip assembly 310 or any other material which is non-reactive or resistant to the product being dispensed, such as various metals, plastics, ceramics, composites, or the like. Additionally, or alternatively, either the plug 132, the main body portion 312, or both may be elastomeric, such that when the cap is in place either the plug 132, the main body portion 312, or both, may expand and deform somewhat to seal the product delivery passageway 317. The cap 130 is configured to couple to the housing 340, such as by use of a snap-fit attachment, a threaded attachment as shown in FIG. 112, a press or friction fit, one or more hinges, or any other suitable means of attachment. As shown in FIG. 12, the cap 130 may have an inner thread that threadingly mates with an outer thread on the first end of the main body portion 312.

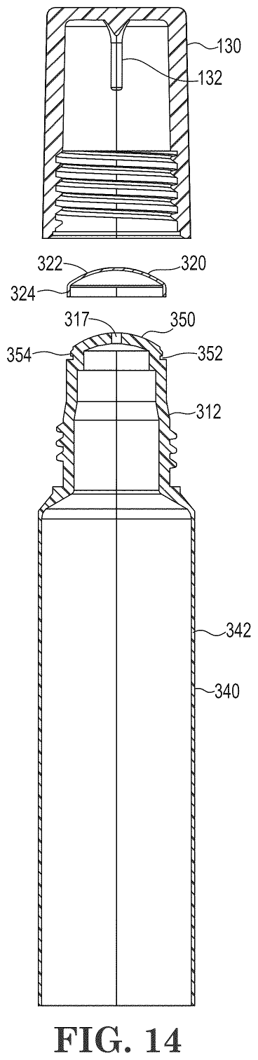

As shown in FIGS. 13 and 14, the metal applicator 320 may have an application face 322 for applying the cosmetic or medicinal product to a surface, and an annular extension or wall 324 extending downward from the application face 322. The metal applicator 320 may define a shell sized and shaped to match an outer surface shape of the upper face 350. The main body portion 312 may further include an annular radially outwardly extending protrusion 354 positioned above the annular groove 352. The protrusion 354 may be configured to engage the wall 324 of the metal applicator 320 in a friction fit or a snap fit. When the metal applicator 320 is fully seated over the upper face 350, an entirety of the upper face 350 may be in contact with an inner surface of the metal applicator 320, such that the product delivery passageway 317 of the main body portion 312 is in contact with and is fluidly coupled to the aperture 131 in the metal applicator 320 to convey the cosmetic or medicinal product from the reservoir 342 defined by the housing 340 to exit the application face 322 of the metal applicator 320. The main body portion 312 may be made of a thermoplastic polymer, such as, for example, polypropylene, which is non-reactive with the product stored in the reservoir 342. In other implementations, the main body portion 312 may be made of plastic, metal, glass, or any other suitable material. The metal applicator 320 may completely cover and be in direct contact with the upper face 350 and protrusion 354 of the second end of the main body portion 312. As with other examples, the seam between the metal applicator 320 and the main body portion 312 is recessed so that the user applying a product will not feel the seam when using the application face 322.

The main body portion 112, 212, 312 may be formed with the housing 140, 240, 340 as a single piece, monolithic structure. In other embodiments, the main body portion 112, 212, 312 may be secured to the housing 140, 240, 340 by, for example, a press-fit, snap-fit, adhesive, threaded fastener, and/or engagement by one or more engagement features. As such, any engagement features on the main body portion 112, 212, 312 may be appropriately designed and configured to enable any, or a combination of, the aforementioned means of attachment. The applicator tip assembly 110, 210, 310 may be fixed to the housing 140, 240, 340 against movement relative to the housing. In other embodiments, the connection between the main body portion 112, 212, 312 and the housing 140, 240, 340 may be flexible such that lateral pressure applied to the second end of the main body portion 112, 212, 312 such as on the application face 122, 222, 322 results in slight bending of the second end of the main body portion with the coupled metal applicator 120, 220, 320 relative to the housing 140, 240, 340.

The delivery mechanism for dispensing the product may be a squeeze operation. When pressure is applied to the housing 140, 240, 340 containing the reservoir 142, 242, 342, the product in the reservoir may be forced, by the squeezing action, through the applicator device 100, 200, 300 via a product delivery passageway 117, 217, 317 and the aperture 121, 221, 321 in the metal applicator 120, 220, 320 for application to the user's skin. Other delivery mechanisms, including pumps that rely on actuation of a button, click mechanism, or dial, may be used instead. A piston may be advanced through the housing 140, 240, 340 using a twist action, for example, to for product out.

While the applicator devices with applicator tip assemblies shown in FIGS. 1-14 are comprised of a single piece applicator tip assembly and housing, in other implementations to decrease manufacturing costs, or for any other desired reason, some or all of the applicator tip assembly and some or all of the housing may be formed separately. In still other implementations, application devices or implements may be configured with an applicator tip assembly as disclosed herein to apply cosmetic product, while omitting a reservoir for holding the cosmetic product. For instance, such an application device or implement may be dipped in a pot or reservoir of product to pick up the product for application to the skin or other surface.

### CONCLUSION

Although the invention has been described in language specific to structural features and/or methodological acts, it is to be understood that the invention is not necessarily limited to the specific features or acts described. Rather, the specific features and acts are disclosed as illustrative forms of implementing the invention.

What is claimed is:

1. An applicator device comprising:
a housing having a reservoir for containing a cosmetic or medicinal product; and
an applicator tip assembly comprising:
a main body portion having a first end coupled to the housing and a second end including an upper face having an annular groove extending inward toward the second end, the annular groove extending radially between an outer wall of the main body portion and an inner wall, the inner wall defining a product delivery passageway configured to convey the cosmetic or medicinal product; and
a metal applicator coupled to the main body portion and having an aperture defined therein, the metal applicator having an application face for applying the cosmetic or medicinal product to a surface, and an annular protrusion extending from a face opposite the application face, the annular protrusion configured to engage the annular groove of the main body portion with a friction fit, the metal applicator having a first outer diameter at the application face and a second, smaller outer diameter at an end opposite the application face;
wherein the product delivery passageway of the main body portion is fluidly coupled to the aperture in the metal applicator.

2. The applicator device of claim 1, wherein the metal applicator defines a radially outwardly extending flange configured to extend over and cover an upwardly facing surface of the outer wall of the second end of the main body portion.

3. The applicator device of claim 1, wherein the upper face of the main body portion is angled relative to a longitudinal axis of the housing.

4. The applicator device of claim 1, wherein the annular protrusion of the metal applicator includes a radially outward extending ridge configured to engage a recess in an inner surface of the outer wall.

5. The applicator device of claim 1, wherein the annular protrusion has a friction fit with at least one of the outer wall and the inner wall.

6. The applicator device of claim 1, wherein the application face has a teardrop shape with the aperture positioned off-center.

7. The applicator device of claim 1, wherein the main body portion comprises a thermoplastic polymer.

8. The applicator device of claim 7, wherein the thermoplastic polymer comprises polypropylene.

9. The applicator device of claim 1, wherein the main body portion and the housing are formed as a single piece.

10. The applicator device of claim 1, wherein the application face is generally convex.

11. The applicator device of claim 1, wherein the applicator tip assembly is fixed to the housing against movement relative to the housing.

12. The applicator device of claim 1, wherein the product delivery passageway of the main body portion ends at a lower surface of the aperture through the metal applicator.

13. The applicator device of claim 1, further comprising a cap configured to removably engage the applicator tip assembly, the cap including a plug that extends into and seals the product delivery passageway when the cap is disposed on the applicator tip assembly.

14. An applicator device comprising:
a housing having a reservoir for containing a cosmetic or medicinal product; and
an applicator tip assembly comprising:
a main body portion having a first end coupled to the housing and a second end opposite the first end, the second end including an outer circumferential wall and an inner post spaced apart from the outer circumferential wall by an annular groove, the inner post defining a product delivery passageway to convey the cosmetic or medicinal product from the reservoir through the main body portion; and
a metal applicator coupled to the main body portion, the metal applicator having an application face for applying the cosmetic or medicinal product to a surface, the metal applicator having an aperture extending therethrough, wherein the aperture is positioned over the inner post and forms a secondary product delivery passageway aligned with the product delivery passageway in the inner post, the metal applicator having a radially extending flange extending over and completely covering the outer circumferential wall of the main body portion.

15. The applicator device of claim 14, wherein the metal applicator includes an annular extension extending away from the application face and configured to be received within the annular groove of the main body portion.

16. The applicator device of claim 15, wherein an outer wall of the annular extension of the metal applicator includes a radially outward extending ridge configured to engage a recess in an inner surface of the outer circumferential wall.

17. The applicator device of claim 15, wherein an outer surface of the inner post includes a radially outward extending ridge configured to engage an inner wall of the annular extension.

18. The applicator device of claim 15, wherein the annular extension has a friction fit with both the outer circumferential wall and an outer surface of the inner post.

19. The applicator device of claim 14, wherein the second end of the main body portion is angled relative to a longitudinal axis of the housing.

20. An applicator device comprising:
a housing having a reservoir for containing a cosmetic or medicinal product; and
an applicator tip assembly comprising:
a main body portion coupled to the housing, the main body portion having a first end coupled to the housing and a second end opposite the first end, the second end defining a tubular product delivery passageway surrounded by an annular channel and an outer wall spaced apart from the tubular product delivery passageway by the annular channel; and
a metal applicator including an application face and an opposing annular wall, the annular wall configured to surround and engage the tubular product delivery passageway in a friction fit, the annular wall configured to be disposed within the annular channel of the main body portion, the metal applicator including a radially outwardly extending flange configured to cover an end face of the outer wall of the main body portion, the application face having an aperture extending therethrough, wherein an upper end of the tubular product delivery passageway fluidly engages a lower end of the aperture.
